(19) United States
(12) Patent Application Publication
(10) Pub. No.: US 2014/0031753 A1
(43) Pub. Date: Jan. 30, 2014

(54) THORACOSTOMY DEVICES AND METHODS OF USE

(71) Applicant: Neil T. Winthrop, West Palm Beach, FL (US)

(72) Inventor: Neil T. Winthrop, West Palm Beach, FL (US)

(21) Appl. No.: 13/951,379

(22) Filed: Jul. 25, 2013

Related U.S. Application Data
(60) Provisional application No. 61/675,392, filed on Jul. 25, 2012.

(51) Int. Cl. A61M 39/10; A61B 17/08

(52) U.S. Cl. CPC A61M 39/10; A61B 17/085
USPC 604/174; 606/214

(57) ABSTRACT

Thoracostomy devices and methods of use are provided herein. In some embodiments, a device may include a seal pad, a petroleum jelly augmented material disposed on the seal pad, a slit formed through both the seal pad and the petroleum jelly augmented layer, the slit being configured to receive and overlap at least a portion of a chest tube extending therethrough, and a securement member extending from an edge of the seal pad, the securement member having a surface that is provided with an adhesive, the securement member being configured to wrap around at least a portion of the chest tube to prevent removal of the chest tube from a patient.

### CROSS REFERENCE TO RELATED APPLICATIONS

This non-provisional U.S. patent application claims the priority benefit of U.S. Provisional Application Ser. No. 61/675,392, filed on Jul. 25, 2012, which is hereby incorporated by reference herein in its entirety including all references cited therein.

### FIELD OF THE INVENTION

The present technology relates generally to thoracostomy devices and methods of use, and more specifically, but not by way of limitation, to thoracostomy support devices that secure a chest tube in situ to reduce infection, prevent undesirable leakage, and/or incidental and undesirable removal of the chest tube from the patient. Additionally, the present technology also provides for an incision sealing device that effectively seals thoracostomy related openings on a patient, such as those created when a chest tube is removed from the body of the patient.

### SUMMARY

According to some embodiments, the present technology may be directed to a medical tube support device, comprising: (a) a seal pad; (b) a petroleum jelly augmented material disposed on the seal pad; (c) a slit formed through both the seal pad and the petroleum jelly augmented layer, the slit being configured to receive and overlap at least a portion of a chest tube extending therethrough; and (d) a securement member extending from an edge of the seal pad, the securement member having a surface that is provided with an adhesive, the securement member being configured to wrap around at least a portion of the chest tube to prevent removal of the chest tube from a patient.

According to some embodiments, the present technology may be directed to a medical tube support device, comprising: (a) a base layer comprising a first surface that is coated with an adhesive; (b) a first layer of absorbent material applied to the surface of the base layer having the adhesive; (c) a second layer that includes a foam material that is impregnated with any of a petroleum jelly and a medicament, the second layer being applied to the first layer; (d) an aperture formed through the base, first, and second layers, the aperture being configured or sized to overlap a chest tube in such a way that the second layer creates a seal around the chest tube; and (e) a slit extending through the base, first, and second layers that allows the chest tube to pass into the aperture.

According to some embodiments, the present technology may be directed to an incision sealing device, comprising: (a) a base layer comprising a first surface that is coated with an adhesive; (b) a first layer of absorbent material applied to the surface of the base layer having the adhesive; and (c) a second layer that includes a foam material that is impregnated with any of a petroleum jelly and a medicament, the second layer being applied to the first layer.

### BRIEF DESCRIPTION OF THE DRAWINGS

Certain embodiments of the present invention are illustrated by the accompanying figures. It will be understood that the figures are not necessarily to scale and that details (e.g., dimensions) not necessary for an understanding of the invention or that render other details difficult to perceive may be omitted. It will be understood that the invention is not necessarily limited to the particular embodiments illustrated herein.

FIG. 1 is a perspective view of an exemplary thoracostomy support device and an exemplary protective covering substrate shown in spaced apart relationship to one another, these devices being constructed in accordance with the present disclosure;

FIG. 2 is an exploded elevational view of various components of the exemplary thoracostomy support device of FIG. 1;

FIG. 3 is a side elevational view of the exemplary thoracostomy support device in an installed configuration on a patient;

FIGS. 4A-C collectively illustrate another exemplary thoracostomy support device;

FIG. 5A is a perspective view of an exemplary incision sealing device and an exemplary protective covering substrate shown in spaced apart relationship to one another, these devices being constructed in accordance with the present disclosure;

FIG. 5B is an exploded front elevational view of various components of the exemplary incision sealing device of FIG. 5A;

FIG. 5C is an exploded side elevational view of various components of the exemplary incision sealing device of FIG. 5A;

FIG. 5D is a perspective view of an exemplary incision sealing device and an exemplary protective covering substrate and adhesive layer shown in spaced apart relationship to one another, these devices being constructed in accordance with the present disclosure;

FIG. 6 is a side elevational view of the exemplary incision sealing device in an installed configuration on a patient;

FIG. 7 is a perspective view of an exemplary thoracostomy support device having a securement member.

FIG. 8A and 8B collectively illustrate another exemplary thoracostomy support device, where FIG. 8B illustrates the removal of sections of a protective layer from the exemplary thoracostomy support device.

### DETAILED DESCRIPTION OF EXEMPLARY EMBODIMENTS

While this invention is susceptible of embodiment in many different forms, there is shown in the drawings and will herein be described in detail several specific embodiments with the understanding that the present disclosure is to be considered as an exemplification of the principles of the invention and is not intended to limit the invention to the embodiments illustrated.

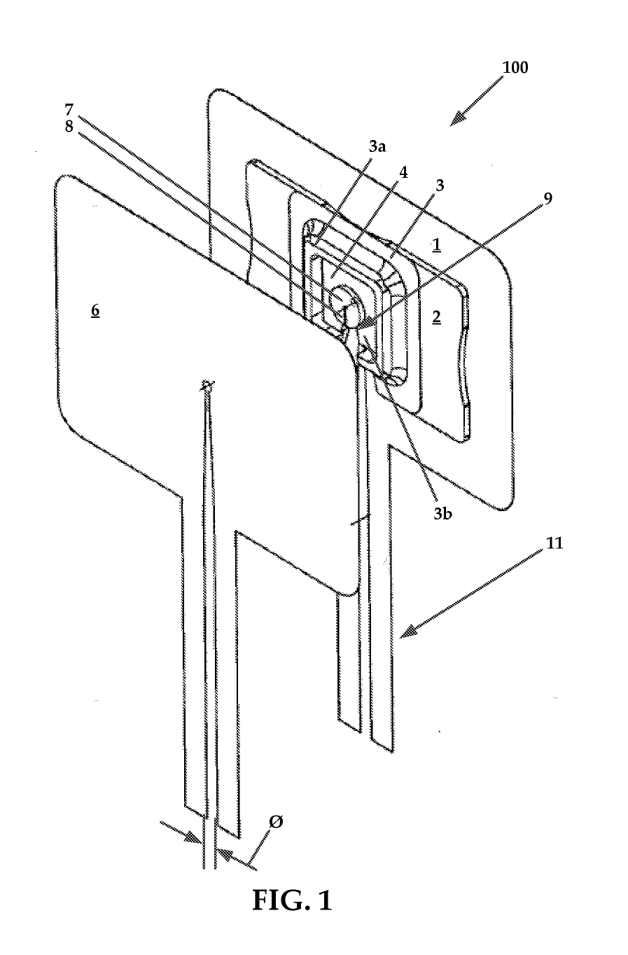

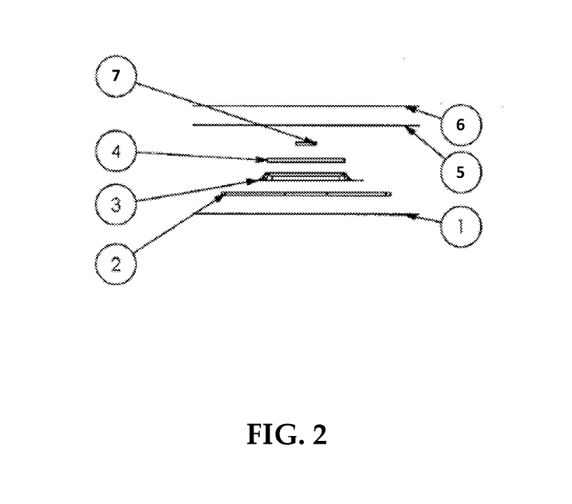

Referring now to the collective drawings, and more particularly to FIGS. 1-2 collectively, which include various views of an exemplary thoracostomy device, hereinafter “device 100” and an exemplary protective covering substrate 6 shown in spaced apart relationship to one another.

Generally speaking, the device 100 may be adapted to secure a chest tube in situ relative to the chest wall of a patient. That is, when thoracostomy procedure is performed, an incision is created in the chest wall of the patient. A chest tube (also known as a chest drain) is then inserted into the incision site. To secure the chest tube in place, the physician will often build a makeshift support device from a trimmed gauze pad which may be provided with petroleum jelly. The makeshift device may also comprise a one or more trimmed layers of sterile gauze. The makeshift support device may be held in place or secured to the chest wall of the patient using tape. In some instances, the gauze pad having petroleum jelly may be wrapped around the outer peripheral sidewall of the chest tube to create a seal between the chest tube and the incision site. That is, the incision site may be larger in length that the diameter of the chest tube, thus creating a pathway for fluid leakage. If this gap is not filled, the likelihood of deleterious effects increases.

These makeshift support devices suffer from a variety of drawbacks, namely the potential for contamination and improper fitting. Generally, medical devices and implements are packaged in sterile packaging such as bags, pouches, or other containers. The sterile environment of the packaging is only breached when the device is needed. Creating makeshift implements on the fly may result in contamination of the component parts of the device because each component part may require trimming. The act of cutting or otherwise modifying a sterile device may impart particulates or contamination from the cutting implement, such as a pair of scissors. Additionally, building a makeshift device requires the physician to handle each component part more frequently than a device that is removed from its sterile packaging and immediately utilized.

Thus, the device 100 of the present technology reduces and eliminates these drawbacks, as will be described in greater detail herein. Generally speaking, the device 100 may comprise a seal pad substrate 1. The seal pad substrate 1 may comprise any suitable material such as a surgical silk or mesh material, although other fluid impermeable materials that would be known to one of ordinary skill in the art with the present disclosure before them are likewise contemplated for use in accordance with the present technology. The seal pad substrate 1 may comprise any thickness or geometrical configuration, although in some embodiments the seal pad substrate 1 may comprise a substantially rectangular shape having rounded corners.

The inner surface of the seal pad substrate 1 may comprise a medical grade adhesive material that is configured to attach to the skin of the patient and create a seal that substantially precludes bodily fluids exuding from the incision site from contacting the body of the patient.

In some embodiments, the seal pad substrate 1 may comprise one or more securement strips 11. These securement strips 11 may comprise an adhesive material that allows the securement strips 11 to be wrapped around the chest tube to reduce the likelihood that the chest tube will be undesirably removed from the patient. Thus, the combination of the adhesive on the inner surface of the seal pad substrate 1, which facilitates attachment of the device 100 to the chest wall of the patient, along with the wrapping of one or more of the securement strips 11 around the chest tube, reduces the likelihood that the chest tube will be removed in an unwanted manner.

Applied to the seal pad substrate 1 is a foam pad substrate 2. The foam pad 2 may comprise any material that is configured to absorb fluids such those which would be expected to exude from a thoracostomy incision site. The foam pad 2 may be exchanged for any similar material that would be known to one of ordinary skill in the art. It is noteworthy that the size of the foam pad 2 may be smaller than that of the seal pad substrate 1 to ensure that the adhesive of the seal pad substrate 1 contacts the skin of the patient.

According to some embodiments, the device 100 may comprise a diaphragm 3 that is positioned between the foam pad 2 and a petrolatum impregnated material 4. The diaphragm 3 may comprise any medical grade material such as a plastic, polymeric, resin, or composite material. It is noteworthy that the material used to construct the diaphragm 3 may include a durable and resilient material.

The diaphragm 3 may comprise a raised outer peripheral edge 3a that forms a cavity 3b that receives the impregnated material 4. According to some embodiments, the diaphragm 3 may be fluid impervious to reduce the passage of fluid therethrough to the foam pad 2. For example, only excess fluids that escape the raised outer peripheral edge 3a of the diaphragm 3 may be absorbed by the foam pad 2. In some instances, the diaphragm 3 may allow for the passage of fluid to the foam pad 2 via holes, slits, apertures, ports, or other similar features.

The impregnated material 4 may comprise a layer of petrolatum bearing material such as medical grade gauze. In some instances, the impregnated material 4 may be referred to as a petroleum jelly augmented material. In some embodiments, the impregnated material 4 may comprise a layer of foam or a sponge that bears or has been impregnated with petrolatum. Advantageously, when the impregnated material 4 may comprises a layer of foam or a sponge, such material may more readily conform to the outer sidewall of the chest tube as compared to a layer of gauze, as will be described in greater detail below. In some instances, the impregnated material 4 may be impregnated with a medicament, and with or without the petrolatum. In additional embodiments, the impregnated material 4 may instead comprise a dry (not impregnated) material that sealingly conforms to the outer sidewall of the chest tube.

According to some embodiments, the impregnated material 14 may comprise a sponge material, which may be constructed from a foam-based or non-adhesive hydrocellular polyurethane apertured dressing having a petrolatum material impregnated therein.

The device 100 may also comprise a protective disk 7 that is layered upon the impregnated material 4. The protective disk 7 may comprise any material, composition, compound, and/or medicament that may reduce catheter-related blood stream infections, such as chlorhexidine gluconate (CHG). The protective disk 7 may comprise an annular ring of material that is sized to receive a standard sized chest tube. It will be understood that size of the protective disk 7 for the device 100 may vary depending upon the desired application (e.g., adult, pediatric, neonatal, etc.). In some instances the disk includes a split 8 that cooperates with the slit formed in the device 100, which will be described in greater detail below. The chest tube is received by the disk 7 by passing the chest tube through the split 8.

While the protective disk 7 has been disclosed as being disk-shaped, it will be understood that the protective disk 7 may comprise any suitable configuration that would be known to one of ordinary skill in the art.

The device 100 may also comprise an adhesive substrate 5 that releaseably secures a backing substrate 6. In some instances, the adhesive substrate 5 may be combined with the backing substrate 6. The backing substrate 6 may cover the various layers of the device 100 to prevent contamination of the same. The backing substrate 6 may comprise any suitable material that would be known to one of ordinary skill in the art.

In some instances, each of the one or more securement strips 11 may be provided with a protective strip of material that protects the adhesive material disposed on the one or more securement strips 11.

In some instances, the device 100 may comprise a slit 9 that extends from between the one or more securement strips 11 all the way up through each of the components of the device 100. The slit 9 allows for the device to overlap the chest tube and sealingly mate with the same, as will be described in greater detail below. In some embodiments, the slit 9 may be wedge shaped, forming an angle . The size of the angle vary according to the diameter of the chest tube. In other embodiments, the slit 9 may be substantially rectangular. Additional embodiments are shown in Appendix A, which is attached hereto and incorporated by reference herein in its entirety.

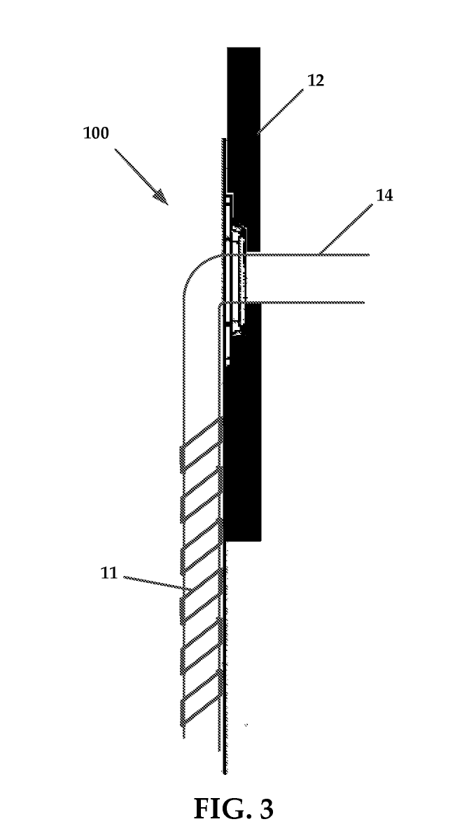

FIG. 3 is a side elevational view of the device 100 in an installed configuration on a chest wall 12 of a patient. In operation, the device 100 may be installed by placing chest tube 14 through the slit 9 of the device 100. As the chest tube 14 is draw up through the slit 9, the foam pad 2, diaphragm 3, and the impregnated material 4 may conform to the outer sidewall of the chest tube 14 to create a seal between the chest tube 14 and the chest wall 12. Once the chest tube 14 engages with protective disk 7 the backing substrate 6 may be removed from the device 100 to expose the adhesive material that is disposed on the inner surface of the seal pad substrate 1.

Once the adhesive layer is exposed, the device 100 may be compressively applied to the chest wall 12 of the patient to secure the device 100 to the chest wall 12. To further secure the chest tube 14, the protective backing (e.g., shown as strips attached to the backing substrate 6 in FIG. 1) may be removed from the one or more securement strips 11. The one or more securement strips 11 may then be wrapped around the chest tube 14 to secure the chest tube 14 in place. Thus, the securement strips 11 may prevent the chest tube 14 from dislodging or being pulled out in an undesirable manner.

It is noteworthy that in some embodiments, various components of the device 100 may be omitted. For example, the device 100 may comprise the seal pad substrate 1, the foam pad 2, and the impregnated material 4, or alternatively, only the seal pad substrate 1 and the impregnated material 4.

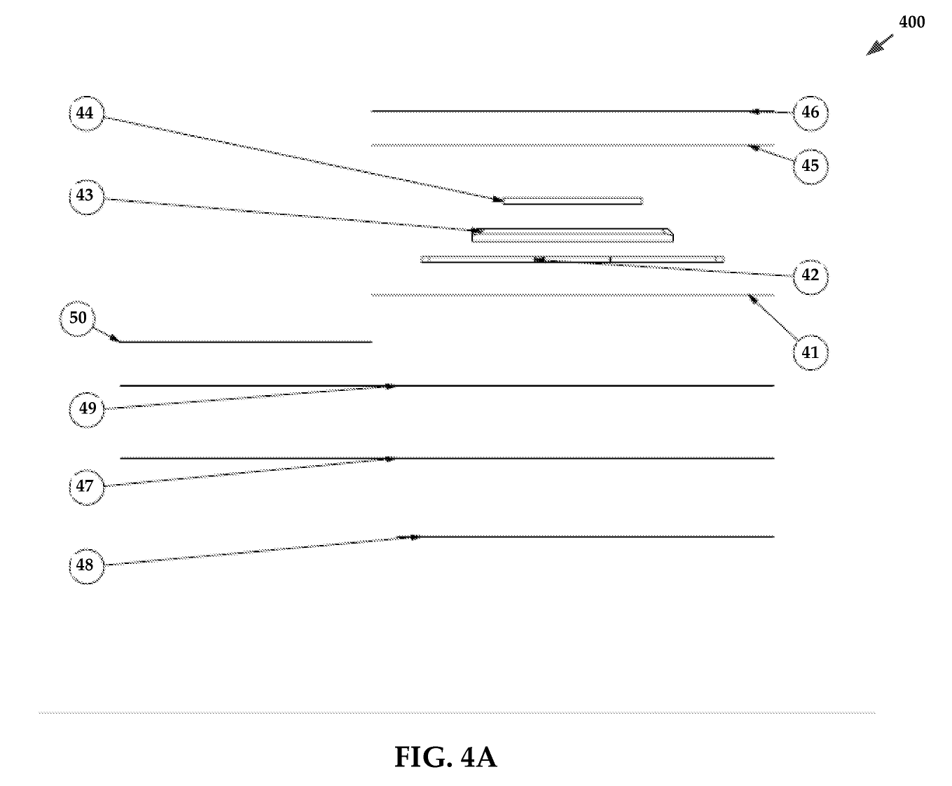

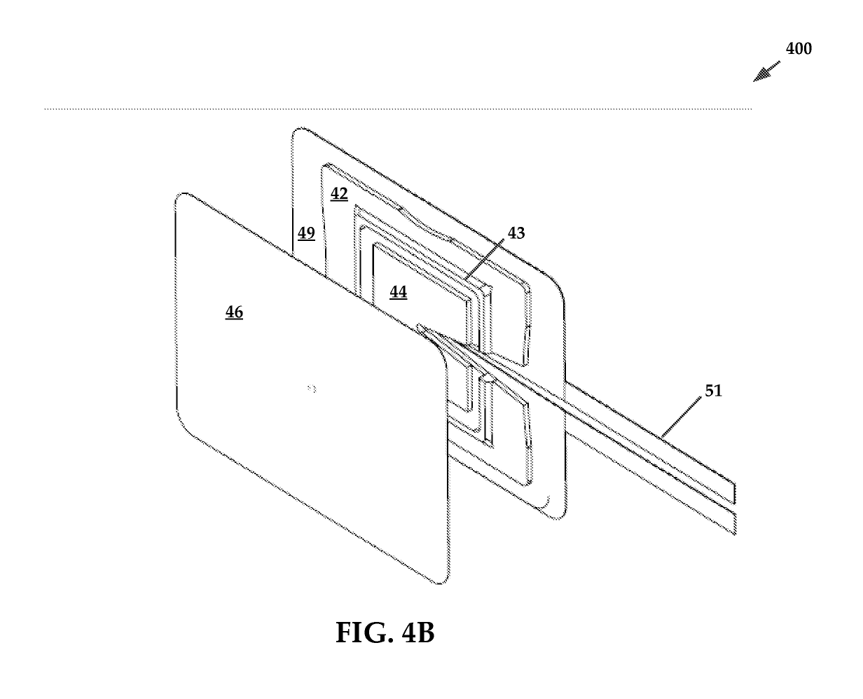

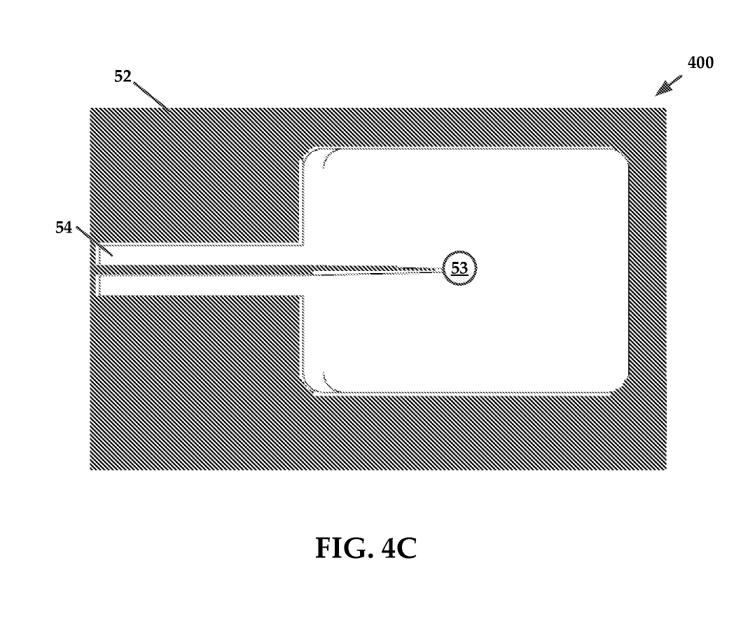

For example, FIG. 4A illustrates an alternative embodiment of the device 100, hereinafter device 400, which comprises a seal pad substrate 41, a foam or gauze pad 42, a diaphragm 43, an impregnated material 44, a double sided adhesive layer 45, and an optional transparent backing layer 46.

The device 400 may also include a layer of tube tape 47, another layer of a backing material 48, a layer of double sided adhesive 49, which is disposed on the backing material 48, as well as a layer of tube tape backing material 50.

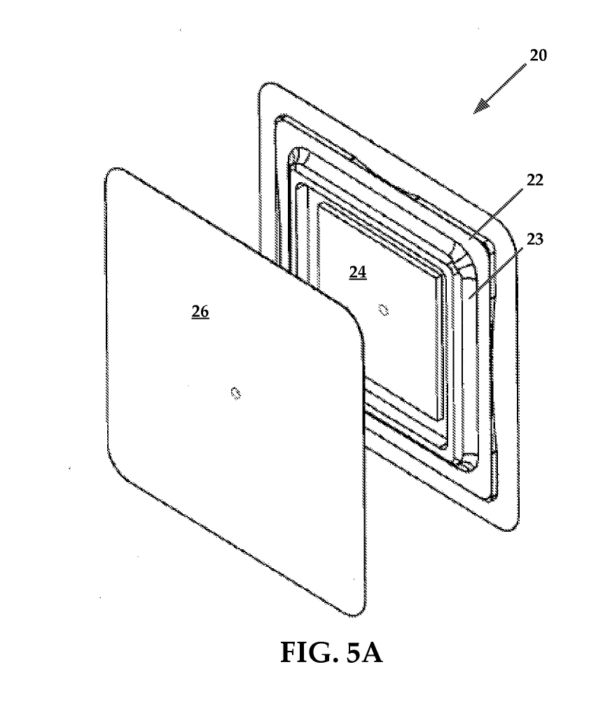

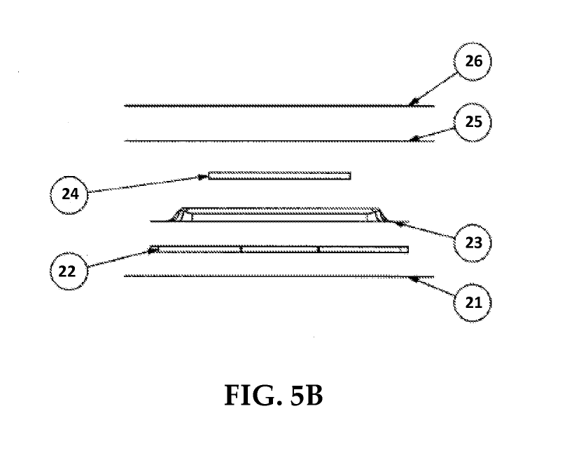

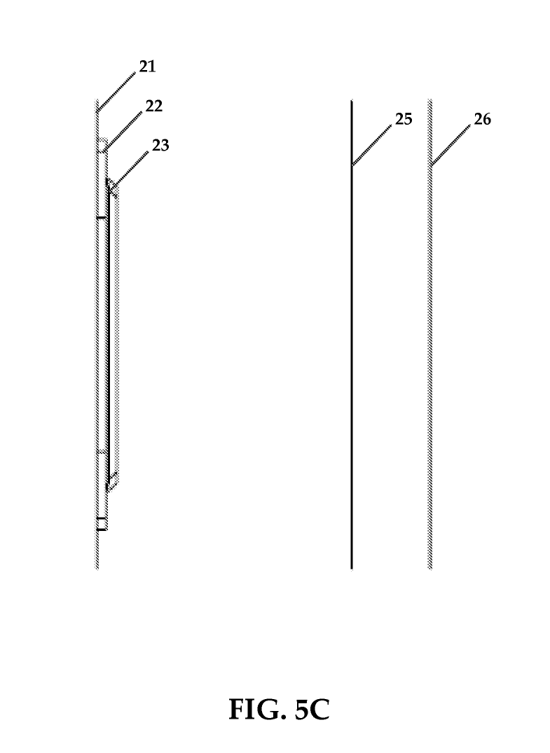

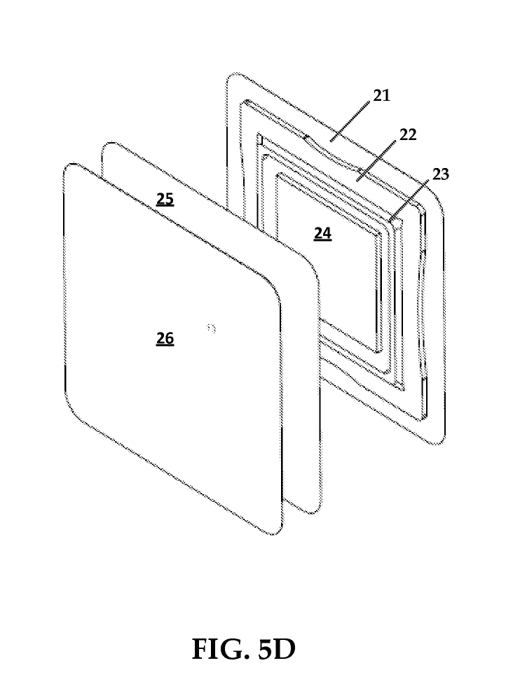

It will be understood that the tube tape layer 47 and the double sided adhesive layer 49 comprise the securement strips 50, as shown in FIG. 5. The tube tape backing 50 is applied to the double sided adhesive layer 49 and is removed when needed.

With reference to FIGS. 4A-C collectively, the device 400 is disposed with the seal pad substrate 41 contacting a chest wall 12 of a patient, similarly to how device 100 is shown as placed against a chest wall 12 in FIG. 3. The chest tube 53 is brought to engagement with the impregnated material 44 by sliding the chest tube 14 along the slit 54. Once the chest tube 53 mates properly with the device 400, the seal pad substrate 41 may be adhered to the chest wall 52. The chest tube 53 may be laid substantially flat along the backing substrate 48 while the layer of adhesive material 49 of the transparent backing layer 46 is exposed. Folding the transparent backing layer 46 over the chest tube 53 secures the chest tube 53 in place. The securement strips 51 may wrapped around the chest tube 53. It is noteworthy that in some instances the two securement strips 51 may be utilized such that the securement strips 51 are wound around the chest tube 53 in opposing direction. As illustrated in FIG. 4C, the securement strips 51 are shown in an unwrapped configuration (e.g., before being wrapped around the chest tube 53.

While the above described embodiments contemplate the sealing and securement of a chest tube, the present technology may also be utilized to seal and secure any number of tubes, drains, or other similar devices relative to a patient's body.

Referring now to FIGS. 5A-D collectively, which show various views of an exemplary incision sealing device, hereinafter “device 20” and an exemplary protective covering substrate 26 shown in spaced apart relationship to one another.

Broadly described, the device 20 may be utilized to create a fluid impervious seal that covers a thoracostomy incision, such as after the removal of a chest tube. The device 20 is shown as generally comprising a seal pad substrate 21. The seal pad substrate 21 may comprise any suitable material such as a surgical silk or mesh material, although other fluid impermeable materials that would be known to one of ordinary skill in the art with the present disclosure before them are likewise contemplated for use in accordance with the present technology. The seal pad substrate 21 may comprise any thickness or geometrical configuration, although in some embodiments the seal pad substrate 21 may comprise a substantially rectangular shape having rounded corners.

The inner surface of the seal pad substrate 21 may comprise a medical grade adhesive material that is configured to attach to the skin of the patient and create a seal that substantially seals a thoracostomy incision. It is noteworthy that the device 20 is well suited to cover many other incisions such as those associated with a tracheostomy.

The device 20 may also comprise a foam pad 22 and a diaphragm 23 that are constructed similarly to those complementary components of the device 100 of FIGS. 1-3. Similarly, the adhesive substrate 25 and the protective covering layer 26 are similar to those complementary components of the device 100 of FIG. 1.

The device 20 may also comprise an impregnated material 24 that fits within a cavity of the diaphragm 23. The impregnated material 24 may comprise a layer of petrolatum bearing material such as medical grade gauze. In some embodiments, the impregnated material 24 may comprise a layer of foam or a sponge that bears or has been impregnated with petrolatum. Advantageously, when the impregnated material 24 may comprises a layer of foam or a sponge, such material may more readily seal around the outer peripheral edge of the incision, as will be described in greater detail below. In some instances, the impregnated material 24 may be impregnated with a medicament, and with or without the petrolatum. In additional embodiments, the impregnated material 24 may instead comprise a dry (not impregnated) material that sealingly covers the outer peripheral edge of the incision. In general, the area of the impregnated material 24 should be of sufficient size to cover a typical incision size (e.g., a length that is somewhat larger than the diameter of the chest tube). Thus, devices 20 of varying size may be manufactured to cover various size incisions, the size of the impregnated material 24 being based at least in part upon a chest tube diameter.

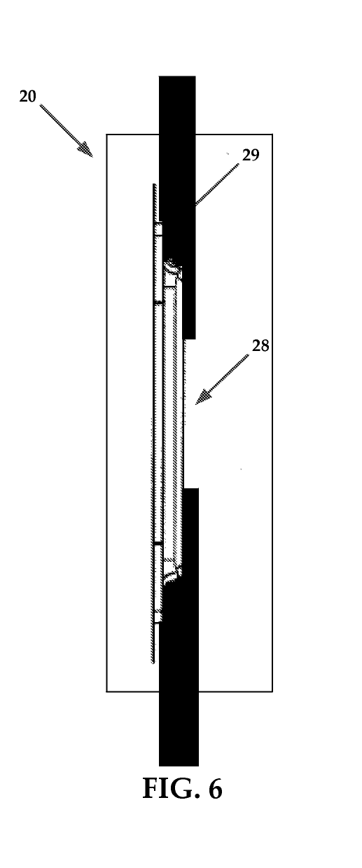

FIG. 6 is a side elevational view of the device 20 in an installed configuration, sealingly covering an incision 28 of a patient. In operation, the protective covering layer 26 is removed from the device 20 exposing the adhesive layer of the seal pad substrate 21.

Next, the impregnated material 24 is disposed above the incision 28 such that the impregnated material 24 covers the incision 28 to prevent the exchange of fluids through the incision 28. The device 20 is pressed against the chest wall 29 of the patient such that the adhesive material of the seal pad substrate 21 contacts the skin of the chest wall 29.

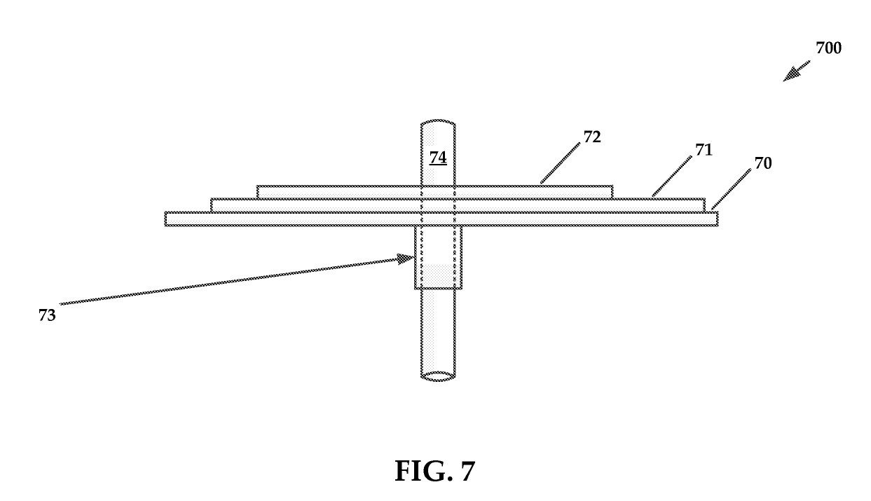

FIG. 7 is a perspective view of an exemplary thoracostomy device, hereinafter “device 700” that may be constructed similarly to the devices 100 and 400 (FIG. 4A-C) described in greater detail above. For purposes of brevity, only a seal pad 70, an absorbing layer 71, and a diaphragm 72 are shown, although the device 700 may include any of the other components of devices 100 and 400 of FIGS. 1 and 4A-C, respectively. Rather than utilizing securement strips (or in addition to), the device 700 may comprise a chest tube clamp 73, which locks the chest tube 74 in place. The clamp 73 may extend normally to the device 700 and may be fixedly attached to the device 700. The clamp 73 may releaseably lock the chest tube 74 in place by exerting compressive forces on the sidewall of the chest tube when the chest tube is inserted into the clamp 73. Stated otherwise, the chest tube may “snap” into the clamp 73 to prevent unwanted movement of the chest tube.

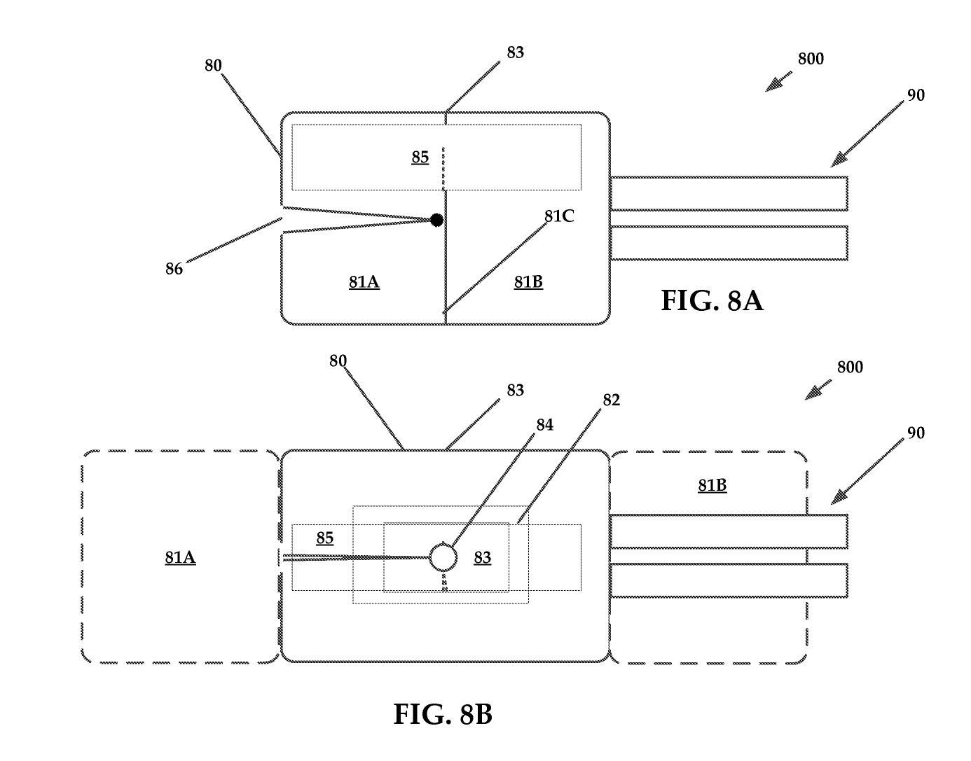

FIGS. 8A-B collectively illustrate an exemplary thoracostomy device, hereinafter “device 800” that may be constructed similarly to the devices 100 and 400 (FIG. 4A-C) described in greater detail above. The device 800 comprises a base layer 80 (e.g., seal pad) and protective layer 81, which is shown as comprising two sections 81A and 81B that can be removed separately from one another. The sections 81A and 81B are divided from one another along a line 81C. This line 81C may include, for example, a perforation or complete separation between sections 81A and 81B.

Thus, in some embodiments, the seal pad 80 (e.g., a base layer) includes a first surface that is coated with an adhesive. Also, the device 800 includes a first layer 82 of absorbent material applied to the surface of the base layer 80 having the adhesive. A second layer 83 that includes a foam material that is impregnated with any of a petroleum jelly and a medicament is applied to the first layer 82.

An aperture 84 is formed through the base 80, first 82, and second 83 layers. The aperture 84 being configured or sized to overlap a chest tube 84 in such a way that the second layer 83 creates a seal around the chest tube 84. Again, a slit 86 extends through the base 80, first 82, and second 83 layers that allows the chest tube 84 to pass into the aperture 84.

In use, the first second 81A is removed, exposing portions of the base 80, first 82, and second 83 layers, and namely the adhesive on the base layer 80. The device 800 is positioned such that the chest tube 84 is drawn through the slit 86 and into the aperture 84, where the second layer 83 enwraps or overlaps the chest tube 84 to create a seal around the chest tube 84. The device 800 is pressed against the incision site on the patient. Next, the second section 81B is removed to expose the rest of the device 800. The exposed remaining section of the device 800 is pressed against the incision site on the patient. Finally, an overlapping layer 85 is folded over to bring the slit 86 closed and cover the remaining portion of the slit 86 to further seal the incision site. The overlapping layer 85 may include a flap of material that is provided with an adhesive, which is covered by a protective layer. The protective layer is removed to expose the adhesive, thus allowing the overlapping layer 85 to bring opposing sides of the base layer 80 on either side of the slit together. Also, the overlapping layer 85 includes a slit 87 that fits around the chest tube 84 to further secure the chest tube 84. It will be understood that rather than having a slit 87, the overlapping layer 85 may include two sections that are at least partially (or not at all) joined together (e.g., shown by dotted line on the overlapping layer 85).

In some embodiments, the securement members 90 are wrapped around the chest tube, as has been described in greater detail supra.

The above description is illustrative and not restrictive. Many variations of the invention will become apparent to those of skill in the art upon review of this disclosure. The scope of the invention should, therefore, be determined not with reference to the above description, but instead should be determined with reference to the appended claims along with their full scope of equivalents.

What is claimed is:

1. A medical tube support device, comprising:
a seal pad;
a petroleum jelly augmented material disposed on the seal pad;
a slit formed through both the seal pad and the petroleum jelly augmented layer, the slit being configured to receive and overlap at least a portion of a chest tube extending therethrough; and
a securement member extending from an edge of the seal pad, the securement member having a surface that is provided with an adhesive, the securement member being configured to wrap around at least a portion of the chest tube to prevent removal of the chest tube from a patient.

2. The device according to claim 1, wherein the slit is substantially wedge shaped.

3. The device according to claim 1, further comprising a layer of absorbent material that is disposed between the seal pad and the petroleum jelly augmented material, wherein the layer of absorbent material has a length and a width that are larger than the petroleum jelly augmented material such to catch fluids that escape around the petroleum jelly augmented material.

4. The device according to claim 3, further comprising a diaphragm that is disposed between the petroleum jelly augmented material and the layer of absorbent material.

5. The device according to claim 4, wherein the slit also formed through the layer of absorbent material and the diaphragm.

6. The device according to claim 4, wherein the diaphragm includes a raised outer peripheral edge that forms a cavity that receives the petroleum jelly augmented material.

7. The device according to claim 1, further comprising another securement member that extends from the seal pad in parallel with the other securement member.

8. The device according to claim 1, wherein the petroleum jelly augmented material includes a non-adhesive hydrocellular polyurethane apertured dressing.

9. The device according to claim 8, further comprising a protective disk that is disposed on top of the petroleum jelly augmented material.

10. The device according to claim 9, wherein any of the protective disk and the petroleum jelly augmented material includes a medicament, wherein the medicament is provided to reduce catheter-related blood stream infections.

11. The device according to claim 9, wherein the protective disk has an annular configuration that includes a split that cooperates with the slit to allow the chest tube to be received by the protective disk.

12. A medical tube support device, comprising:
a base layer comprising a first surface that is coated with an adhesive;
a first layer of absorbent material applied to the surface of the base layer having the adhesive;
a second layer that includes a foam material that is impregnated with any of a petroleum jelly and a medicament, the second layer being applied to the first layer;
an aperture formed through the base, first, and second layers, the aperture being configured or sized to overlap a chest tube in such a way that the second layer creates a seal around the chest tube; and
a slit extending through the base, first, and second layers that allows the chest tube to pass into the aperture.

13. The device according to claim 12, wherein the base, first, and second layers are covered with a protective layer to preserve the adhesive.

14. The device according to claim 13, wherein the protective layer includes two sections that can be removed from the base layer separately from one another.

15. An incision sealing device, comprising:
a base layer comprising a first surface that is coated with an adhesive;
a first layer of absorbent material applied to the surface of the base layer having the adhesive; and
a second layer that includes a foam material that is impregnated with any of a petroleum jelly and a medicament, the second layer being applied to the first layer.

16. The device according to claim 15, further comprising a diaphragm disposed between the first layer and the second layer, wherein the diaphragm includes a raised outer peripheral edge that forms a cavity, the second layer being disposed entirely within the cavity.

17. The device according to claim 15, wherein the medicament includes chlorhexidine gluconate.
